(19) United States
(12) Patent Application Publication
(10) Pub. No.: US 2021/0025904 A1
(43) Pub. Date: Jan. 28, 2021

(54) GLYCATED HEMOGLOBIN MEASUREMENT

(71) Applicant: Ortho-Clinical Diagnostics, Inc., Raritan, NJ (US)

(72) Inventors: Gary L. Snodgrass, Pittsford, NY (US); Robert Fazio, Rochester, NY (US); Christine M. Hoffman, Hilton, NY (US); Brian R. Johnson, Webster, NY (US); Glen D. Labenski, Pittsford, NY (US)

(21) Appl. No.: 16/935,867

(22) Filed: Jul. 22, 2020

Related U.S. Application Data
(60) Provisional application No. 62/877,188, filed on Jul. 22, 2019.
(60) Provisional application No. 63/040,159, filed on Jun. 17, 2020.

(51) Int. Cl. G01N 33/72; B01L 3/00; C12Q 1/37

(52) U.S. Cl. CPC G01N 33/723; B01L 3/508; C12Q 1/37; B01L 2300/168; B01L 2300/0822; B01L 2300/12; G01N 2800/042

(57) ABSTRACT

Described herein are devices, systems, and methods used to measure glycated hemoglobin.

### CROSS REFERENCE TO RELATED APPLICATIONS

This application claims the benefit of U.S. Provisional Patent Application No. 63/040,159, filed Jun. 17, 2020, and U.S. Provisional Patent Application No. 62/877,188, filed Jul. 22, 2019, the entire disclosures each of which is incorporated herein by reference.

### FIELD

Described herein are devices, systems, and methods used to measure glycated hemoglobin.

### SUMMARY

Described herein generally are devices, systems and methods for hemoglobin measurement, in particular, measurement of glycated hemoglobin. Determining the level of glycated hemoglobin in a patient sample is a key component in the diagnosis of diabetes mellitus Types I and II and gestational diabetes, because it can estimate an individual's average blood glucose levels over a period of time (e.g., three months).

In some embodiments, this glycated hemoglobin measurement can be of whole blood. The whole blood can be human or animal whole blood. However, measurements can also be made of various components of blood as long as the hemoglobin components are present.

Generally, measurements can be performed using a microslide test element or simply a microslide such as a dry slide test element. These microslides can be used in automated analyzers. The microslides can be single-slides thereby completing the entire analysis using a drop or drops of sample and a single slide, not multiple slides. In some embodiments, multiple measurements can be made on a single slide.

The microslides described herein can include a stack of film layers comprising, from bottom to top, a first film layer comprising a cross-linked gel, wherein said cross-linked gel comprises a detection agent, a fructosyl oxidase, and a peroxidase, a second film layer comprising a first gel, and a third film layer comprising a lysing agent, a denaturing agent and a protease. In some embodiments, the first film layer is a gel layer, the second film layer is a masking layer, and the third film layer is a spread layer. In some embodiments, the microslide can include an adhesion or sub layer between the masking layer and the spread layer.

The microslides described herein can include a stack of film layers comprising, from bottom to top, a first film layer comprising a cross-linked gel, wherein said cross-linked gel comprises a detection agent, a fructosyl oxidase, an interference prevention agent, and a peroxidase, a second film layer comprising a first gel, and a third film layer comprising a lysing agent, a denaturing agent and a protease. In some embodiments, the first film layer is a gel layer, the second film layer is a masking layer, and the third film layer is a spread layer. In some embodiments, the microslide can include an adhesion or sub layer between the masking layer and the spread layer.

The microslides described herein can include a stack of film layers comprising, from bottom to top, a gel layer, a masking layer, and a spread layer. In other embodiments, the microslides described herein can include a stack of film layers comprising, from bottom to top, a gel layer, a masking layer, an adhesion or sub layer, and a spread layer. In some embodiments, a first layer, or the gel layer, comprises a gel. In some embodiments, a second film layer, the masking layer comprises a second gel. In some embodiments, a third film layer, the spread layer comprises a lysing agent, a denaturing agent and a protease. In some embodiments, an adhesion or sublayer is included as a third layer and the spread layer is a fourth layer.

In some embodiments, the gels can be cross-linked gels. Other layers can be included in microslides. In some embodiments, the cross-linked gel comprises a detection agent, a fructosyl oxidase, and a peroxidase.

The first film layer can further comprise an oxidase cofactor and a surfactant. In some embodiments, the oxidase cofactor is flavin adenine dinucleotide (FAD). The fructosyl oxidase can be specific for a Fru-α-ValHis peptide or a glycated amino acid such as Fru-α-Val. In some embodiments, interference prevention agent is ascorbic acid oxidase (AAO).

In some embodiments, the detection agent is a leuco-dye, such as a blue leuco-dye. The detection agent can be selected from the group consisting of N-carboxymethylaminocarbonyl)-4,4′-bis(dimethylamino)-diphenylamine sodium (DA-64), N,N,N′N′, N″,N″-hexa(3-sulfopropyl)-4,4′,4″-triamino-triphenylmethane hexasodium salt (TPM-PS), 10-(carboxymethylaminocarbonyl)-3,7-bis(dimethylamino)-phenothiazine sodium (DA-67), and 2-(3,5-dimethoxy-4-hydroxyphenol)-4,5-bis-(4-dimethylamino phenyl) imidazole.

In some embodiments, the peroxidase is horseradish peroxidase.

In some embodiments, the second film layer further comprises a reflective material portion. The reflective material portion can comprise a metal salt. In one embodiment, the metal is titanium such as, but not limited to titanium dioxide (TiO₂).

In some embodiments, the third film layer further comprises calcium.

In some embodiments, the third film layer comprises a porous layer containing latex particles. In some embodiments, the latex particles can be formed of vinyltoluene-co-methacrylic acid copolymer (VtE). The particles, or sometimes referred to as beads, can have a median particle size of about 25 μm. In one embodiment, the particles can have a median particle size of less than 25 μm. In other embodiments, the particles can have a median particle size of about 10 μm to about 40 μm, about 15 μm to about 35 μm, about 20 μm to about 30 μm, less than about 15 μm, less than about 10 μm, or less than about 5 μm.

In some embodiments, the lysing agent is a detergent. The detergent can be selected from the group consisting of octylphenol ethoxylate (TRITON® X-100, Union Carbide Corporation, New York), TWEEN® (ICI Americas Inc., Delaware) (TWEEN 20), sodium dodecyl sulfate (SDS), cetyltrimethylammonium bromide (CTAB), tetradecyltrimethylammonium bromide (TTAB), polyoxyethylene lauryl ethers (POEs) and NONIDET® (Air Products and Chemicals, Inc., Delaware) P-40 (NP-40). In one embodiment, the detergent is TRITON X-100.

In some embodiments, the denaturing agent is an oxidant or a surfactant. The denaturing agent can be one or more of sodium nitrite or N-lauroylsarcosine (NLS).

In some embodiments, the protease is a metalloproteinase and/or a neutral protease. The protease can be an endoprotease or an exoprotease. In other embodiments, the protease is selected from the group consisting of proteinase K, pronase E, protease XVII, protease XXI, an aminopeptidase, a carboxypeptidase, thermolysin, bacillolysin, a microbial metalloproteinase, peptidase K, endoproteinase K, chymotrypsin, chymotrypsin C, glutamyl endopeptidase, peptidyl-lys-metalloendopeptidase, protease from Bacillus sp., leucyl aminopeptidase, and subtilisin.

In some embodiments, the third film layer is in direct contact with an upper surface of the second film layer. In other embodiments, the second film layer is in direct contact with an upper surface of the first film layer.

In some embodiments, the spread layer is in direct contact with an upper surface of the masking layer. In other embodiments, the spread layer is in direct contact with an upper surface of the adhesion layer.

Also described herein are single-slide methods for detecting hemoglobin and glycated hemoglobin. These methods comprise a) providing a microslide as described herein; b) contacting the third film layer of the microslide with an untreated blood sample comprising red blood cells, wherein the lysing agent releases glycated hemoglobin from the red blood cells, wherein the denaturing agents contact the glycated hemoglobin to denature the glycated hemoglobin, and wherein the protease releases a fructosyl peptide from the denatured glycated hemoglobin, wherein the fructosyl peptide reaches the first film layer and reacts with the fructosyl oxidase and FAD cofactor to generate peroxide, and wherein the peroxidase and the peroxide react with the detection agent to release a detectable signal; c) measuring the amount of hemoglobin from the blood sample, wherein the measuring the amount of hemoglobin comprises measuring the reflectance density of the sample from said slide at a first wavelength of light; and d) measuring the amount of glycated hemoglobin from the blood sample, wherein the measuring the amount of glycated hemoglobin comprises measuring the reflectance density from the detection agent at a second wavelength of light, wherein the second wavelength of light is different from the first wavelength of light. In some embodiments, the detection agent is an oxidized dye.

In some embodiments, the untreated blood sample can be an un-lysed blood sample. In some embodiments, the untreated blood sample can be blood that has been subjected to a clotting prevention agent, but not a lysing agent. In some embodiments, the clotting prevention agent is an anticoagulant. In some embodiments, the untreated blood can be whole blood.

In some embodiments, the first wavelength of light is 540 nm and the second wavelength of light is 670 nm.

In some embodiments, the methods further comprise contacting the fructosyl peptide with an oxidase cofactor such as a flavin adenine dinucleotide (FAD).

Also described herein are single-slide methods for direct detection of glycated hemoglobin. These methods comprise a) providing a microslide wherein the second film layer comprises a reflective material portion; b) contacting the third film layer of the slide with a blood sample comprising untreated red blood cells, wherein the lysing agent releases glycated hemoglobin from the red blood cells, wherein the denaturing agents contact the glycated hemoglobin to denature the glycated hemoglobin, and wherein the protease releases a fructosyl peptide from the denatured glycated hemoglobin, wherein the fructosyl peptide traverses the second film layer, wherein the fructosyl peptide reaches the first film layer and reacts with the fructosyl oxidase and FAD cofactor to generate peroxide, and wherein the peroxidase and the peroxide react with the detection agent to release a detectable signal; and c) measuring the amount of glycated hemoglobin from the blood sample, wherein the measuring the amount of glycated hemoglobin comprises measuring the reflectance density of the oxidized dye in the blood sample.

In some embodiments, the reflective material portion comprises a metal salt.

The whole blood sample can traverse the third film layer due to the porosity created by the latex particles in this layer.

### BRIEF DESCRIPTION OF THE DRAWINGS

FIG. 1 illustrates layers incorporated into a microslide as described herein.

FIG. 2 illustrates a comparison of slides including TiO₂ in their masking layer and slides without TiO₂ in their masking layers.

FIGS. 3A and 3B illustrate 540 nm signal for a non-TiO₂ containing masking layer (“Control”) and a TiO₂ containing masking layer.

FIGS. 4A and 4B illustrate 670 nm signal for a non-TiO₂ containing masking layer (“Control”) and a TiO₂ containing masking layer.

FIG. 5 illustrates dose response for Fru-VH substrate in the presence of increasing hemoglobin (Hb) concentrations for slides not containing TiO₂ in their masking layers.

FIG. 6 illustrates dose response for Fru-VH substrate in the presence of increasing hemoglobin (Hb) concentrations for slides containing TiO₂ in their masking layers.

FIGS. 7A-7D illustrate kinetic data for % A1c model fluids and % A1c patient samples at 670 nm over a 5 minute time period on an analyzer at 37° C. Protease and sodium nitrite are deposited by inkjet and dried on finished microslides.

FIGS. 8A and 8B illustrate kinetic data for % A1c model fluids and % A1c patient samples at 670 nm over a 5 minute time period on an analyzer at 37° C. All components are incorporated by the x-hopper coating process.

FIG. 9 illustrates reflectance density dose response data for % A1c model fluids and % A1c patient samples at 670 nm over a 5 minute time period on an analyzer at 37° C. All components are incorporated by the x-hopper coating process.

FIG. 10 illustrates a correlation plot for microslide % A1c versus BioRad Variant HPLC % A1c.

FIG. 11 illustrates a microslide Hemoglobin component patient sample dose response.

FIG. 12 illustrates a correlation plot for microslide hemoglobin component assay versus MicroTip reference hemoglobin component assay.

FIG. 13 illustrates a microslide HbA1c component patient sample dose response.

FIG. 14 illustrates a correlation plot for microslide HbA1c component assay versus MicroTip reference HbA1c component assay.

FIG. 15 illustrates a correlation plot for microslide derived % A1c assay versus HPLC reference % A1c assay.

FIG. 16 illustrates a HbA1c enzymatic 670 nm dose response plot for the dual assay microslide test element.

FIG. 17 illustrates a Hemoglobin spectra 540 nm dose response plot for the dual assay microslide test element.

### DETAILED DESCRIPTION

The use of a thin film test element, a microslide, to conduct an enzymatic cascade to measure glycated hemoglobin concentration by direct means (% A1c measurement only) or by a derived calculation (HbA1c and hemoglobin measurement to yield a % A1c result) using a patient sample is described. In some embodiments, the patient sample is a whole blood sample such as an untreated whole blood sample. In some embodiments, the untreated blood sample can be an un-lysed blood sample. In some embodiments, the untreated blood sample can be blood that has been subjected to a clotting prevention agent, but not a lysing agent. In some embodiments, the clotting prevention agent is an anticoagulant. In some embodiments, the untreated blood can be whole blood.

The blood sample can be human or animal blood. In some embodiments, the blood can be mammal blood. Mammals can include, but are not limited to humans, horses, camels, dogs, cats, cows, bears, rodents, sheep, goats, pigs and the like. Other animal blood such as reptile, fish, and bird blood can also be used.

In some embodiments, measurements can also be made of various components or fractions of a patient blood sample as long as the hemoglobin components are present.

The measurements are accomplished using devices, systems and methods as described herein. Generally, measurements can be performed using a microslide such as a dry microslide. The microslides can be single-slides thereby completing the entire analysis using a drop of sample and a single slide, not multiple slides.

The herein described microslides can be utilized in an automated analyzer system or other type of mainframe analyzer. These types of instruments can, in some embodiments, process hundreds or even thousands of sample analyses per working day. In one embodiment, the microslides can be used on the current VITROS mainframe analyzers (5,1 FS, 4600 Chemistry System, 5600 Integrated System) and as well as future VITROS analyzers. In addition, the herein described microslides can be used in systems manufactured by Abbott Laboratories, Beckman Coulter, Baxter, Genprobe, Roche Diagnostics, and Siemens.

However, in other embodiments, the microslides can be utilized in non-automated or semi-automated systems. In some embodiments, the microslides can be used in a sample-by-sample scenario and/or loaded by hand.

In some embodiments, microslide test elements described herein can incorporate components of an enzymatic cascade. This enzymatic cascade can result in a generation of a colorimetric signal which relates directly to the concentration of glycated hemoglobin (as % A1c) in a patient sample.

The level of hemoglobin glycation is typically determined in the industry by measuring both hemoglobin (Hb) concentration and glycated hemoglobin (HbA1c) concentration and expressing this as a ratio (derived % A1c). This requires two sets of calibrators to yield separate calibration curves for the determinations for Hb and HbA1c. Alternatively, the assay may be calibrated using known % A1c fluids as calibrators in order to provide the % A1c of the unknown patient sample directly.

The devices, systems, and methods described herein can use microslides that can be utilized in either assay format. In one embodiment, all components necessary to determine the Hb concentration and HbA1c concentration may be incorporated in a single test element to yield a derived % A1c result. In such an embodiment, Hb and HbA1c can each be determined at a different detection wavelength from a single slide, the test can be performed from a single whole blood metering event, and can be performed using current microslide protocols.

In another embodiment, a second option is to use separate microslides to individually measure Hb concentration and HbA1c concentration, respectively to yield a derived % A1c result. In such an embodiment, Hb and HbA1c concentrations can each be determined at a different detection wavelength from individual test slides in a single test element, the test can be performed from two whole blood metering events, and can be performed using current microslide protocols.

Further still, in another embodiment, a single microslide can be used to directly measure % A1c. In such an embodiment, % A1c can be determined at a single detection wavelength from a single microslide. The measurement can be performed from a single whole blood metering event, and can be performed using current microslide protocols.

In some embodiments, the devices, systems, and methods can use an enzymatic cascade to determine HbA1c as % A1c. An enzymatic cascade utilized in a herein described microslide is:

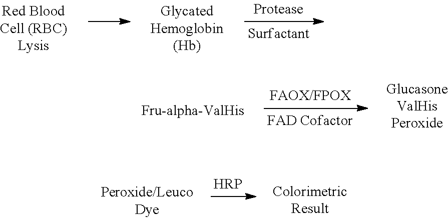

In some embodiments, the cascade can be used for the direct determination of glycated hemoglobin (as % A1c). However, this cascade can also be used when measuring derived % A1c.

Generally, methods of determining % A1c, either direct or derived, include the steps of applying an untreated whole blood sample to a microslide as described herein. In some embodiments, the microslides can include two or more sample locations and can require more than one blood sample.

A lysing surfactant can lyse the red blood cells in the blood sample thereby releasing glycated hemoglobin. A second surfactant can denature the glycated hemoglobin thereby providing access to a proteolytic cleavage site. A protease then cleaves the N-terminal portion of the hemoglobin beta chains thereby releasing a glycated di-peptide (fructosyl-alpha-valyl-histidine-Fru-alpha-ValHis). The glycated di-peptide is then deglycated in an oxidase reaction utilizing a fructosyl peptide oxidase (FPOX) and flavin adenine dinucleotide (FAD) thereby yielding hydrogen peroxide (H₂O₂). The H₂O₂ and a horseradish peroxidase (HRP) oxidize a leuco dye resulting in a colorimetric signal at 670 nm. The concentration of glycated hemoglobin is directly proportional to the reflectance density of the dye formed. In some embodiments, a signal can be read for hemoglobin at 540 nm and a derived % A1c value can be determined utilizing the hemoglobin component determination at 540 nm and the glycated hemoglobin determination at 670 nm.

In some embodiments, a microslide can include at least a first film layer, a second film layer, and a third film layer. A microslide can include more layers. In some embodiments, the first film layer can include a cross-linked gelatin or gel, wherein said cross-linked gel comprises a detection agent, a fructosyl oxidase, a peroxidase, and optionally an interference prevention agent. In some embodiments, the second film layer can include a gelatin, gel, or crosslinked gel or gelatin. In some embodiments, the third film layer can include a lysing agent, a denaturing agent and/or a protease.

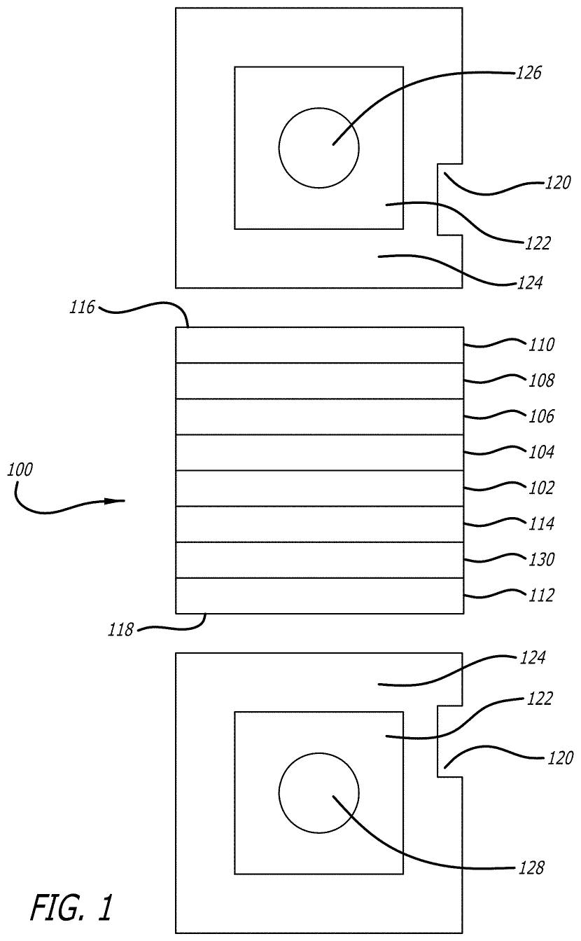

A microslide 100 can include a stack of film layers or simply a stack of layers. The stack of film layers can include a gel layer 102, a masking layer 104, an adhesion layer 106, and a spread layer 108 as illustrated in FIG. 1. In some embodiments, microslide 100 can include an upper slide mount 110, a lower slide mount 112, or both. In some embodiments, when formed, the layers can be built on a support layer 114. In some embodiments, adhesion layer 106, masking layer 104, and gel layer 102 can be combined as a reagent layer.

In some embodiments, support layer 114 can be formed of polyethylene terephthalate or another appropriate transparent polymeric material. This transparent polymeric material allows layers to be applied or coated thereon.

In some embodiments, upper slide mount 110 and lower slide mount 112, are formed of polystyrene or another appropriate polymeric material.

Each layer and mount can be combined to form a microslide that is square or generally rectangular upper surface 116 and lower surface 118. In some embodiments, upper surface 116 and lower surface 118 can have other shapes such as, but not limited to, triangular, pentagonal, hexagonal, heptagonal, octagonal, circular, oval, elliptical, or other rectilinear or circular shape.

In some embodiments, a microslide can include at least one notch or keying surface. A notch or keying surface can be used to assist in stacking multiple microslides and/or loading a microslide(s) into an analyzing instrument. In one embodiment, microslide 100 can include notch 120. Notch 120 is shown as having a rectilinear shape, but in other embodiments, notch 120 can be virtually any shape that allows stacking and/or loading.

Further, microslide 100 can include a window portion 122 on upper surface 116 and/or lower surface 118. Windows portion 122 can be surrounded by a frame portion 124. However, in some embodiments, a frame portion is not included and window portion 122 can extend to a microslide's edge.

Microslide 100 can also include a sample area 126 within window portion 122 on upper surface 116. Sample area 126 can serve as a location for sample application. On lower surface 118, a detection area 128 can exist within window portion 122. Detection area 128 can serve as a location for detection using an analyzer.

Here the layers will be described as a sample travels through the microslide layers. Spread layer 108 can be the first layer that a sample interfaces with. Spread layer 108 can include polymeric beads, a binder, a buffer, at least one surfactant, a divalent cation salt, sodium nitrite, a protease, an alcohol, and water.

In some embodiments, the divalent cation salt can be calcium chloride or any compound that can binds EDTA.

In some embodiments, when forming or applying the spread layer, tert-butyl alcohol can be present. However, it is not present after drying.

In some embodiments, the polymeric beads can include acrylic beads such as but not limited to vinyltoluene-co-methacrylic acid copolymer beads (VtE Beads). The function of the beads can be to create pores in the spread layer that allow red blood cells to enter the coating. The beads can also serve to provide a white reflective surface, promote uniform sample spreading, and trap interferents such as heme byproducts, catalase, triglycerides, and the like.

The beads can have an average diameter that is large enough for red blood cells to penetrate into the coating. In some embodiments, the diameter is greater than about 5 μm, greater than about 10 μm, greater than about 50 μm, greater than about 80 μm, between about 20 μm and about 100 μm, between about 20 μm and about 30 μm, between about 10 μm and about 40 μm, between about 10 μm and about 100 μm, between about 20 μm and about 25 μm, between about 50 μm and about 100 μm, between about 25 μm and about 30 μm, less than about 100 μm, less than about 80 μm, less than about 50 μm, less than about 40 μm, or less than about 30 μm.

The pores created by the beads can have a pore size greater than about 5 μm, greater than about 10 μm, greater than about 20 μm, between about 20 μm and about 30 μm, between about 10 μm and about 40 μm, between about 20 μm and about 25 μm, between about 25 μm and about 30 μm, less than about 50 μm, less than about 40 μm, or less than about 30 μm. In one embodiment, the pore size is about 25 μm.

The binder, which can serve to promote layer cohesion, can be a latex. In one embodiment, the latex can have a percent of solids of the molecular weight of monomer (MWM) latex in the final product of about 30%. In some embodiments, the latex includes a biocide such as but not limited to nipacide. In other embodiments, alternative binders may be utilized such as but not limited to polyacrylamide (l100).

The buffer serves to keep the layer at a desired pH. A desired pH can be about 6.0 to about 7.0, about 6.2 to about 7.2, about 6.5 to about 7.5, about 6.0 to about 8.0, about 7.0 to about 8.0, about 6.8 to about 7.2, about 7.4, about 7.2, about 7.0, or about 6.8. The buffer can be an acid or a base as required. In one embodiment, the buffer is 3-(N-morpholino)propanesulfonic acid (MOPS). Other buffers can include, but are not limited to sodium bicarbonate, calcium carbonate, potassium phosphate, tris(hydroxymethyl)aminomethane (TRIS), Bicine, Bis-TRIS, TES, HEPPS (EPPS), or the like, or combinations thereof.

Some embodiments can include a first surfactant and a second surfactant. The first surfactant can be a lysing agent. The lysing agent can lyse red blood cells and release hemoglobin and glycated hemoglobin. The lysing agent can be a detergent.

In some embodiments, the detergent can be selected from octylphenol ethoxylate (TRITON X-100), TWEEN (TWEEN 20), sodium dodecyl sulfate (SDS), cetyltrimethylammonium bromide (CTAB), tetradecyltrimethylammonium bromide (TTAB), polyoxyethylene lauryl ethers (POEs), NONIDET P-40 (NP-40), or a combination thereof. In one embodiment, the detergent is an octylphenol ethoxylate such as TRITON X-100.

In some embodiments, the second surfactant can be a denaturing agent that denatures hemoglobin. This denaturing agent can expose a site on hemoglobin for proteolysis. The denaturing agent can allow heme oxidation in a single oxidation state and assists in conversion of hemoglobin forms (oxy, deoxy, carboxy) to a single spectral form.

In one embodiment, the denaturing agent can include N-lauroylsarcosine (NLS). In some embodiments, the second surfactant can also include a denaturant aid or oxidant such as sodium nitrite. The denaturant aid may help promote denaturation by the NLS by coordinating with iron in heme.

In one embodiment, the denaturing agent(s) present in the coating can denature the glycated hemoglobin providing accessibility to the protease cleavage site of interest.

In some embodiments, spread layer 108 can include further surfactants as needed to initiate the enzymatic cascade.

Sodium nitrite can be present in molar excess. In some embodiments, the sodium nitrite (NaNO₂) can be present at about 5-10× the total hemoglobin concentration. In some embodiments, the sodium nitrite can act to oxidize heme to a ferric state (+3).

Further, a combination of a denaturing surfactant, sodium nitrite, and hemoglobin can create a single spectral form of hemoglobin which can be read at 540 nm.

The protease can be a neutral protease. The protease can be a metalloproteinase. The protease can be endoprotease or an exoprotease. The protease can generate Fru-alpha-ValHis by cleaving from the N-terminus of a hemoglobin beta subunit or chain. The Fru-alpha-ValHis can be the substrate for the fructosyl oxidase included in the herein described gel layer.

In some embodiments, the protease can be proteinase K, pronase E, protease XVII, protease XXI, an aminopeptidase, a carboxypeptidase, thermolysin, subtilisin, or a combination thereof.

The Fru-α-ValHis dipeptide can be of sufficiently small molecular weight that it can readily pass through the adhesion layer and masking layer and into to the herein described gel layer.

The calcium component can be essential to protease activity. In some embodiments, calcium in the microslide can protect the protease from EDTA anticoagulant in a blood draw tube. In some embodiments, the calcium source is calcium chloride (CaCl₂). In other embodiments, the calcium chloride is calcium chloride di-hydrate.

The calcium chloride present in the spread layer can act to protect protease activity. In some environments, a blood draw tube in the clinical setting for HbA1c measurement is an EDTA plasma tube. Without calcium chloride, the EDTA may bind the calcium and zinc from the protease greatly reducing its proteolytic activity.

The spread layer solvent can be methanol, ethanol, tert-butyl alcohol, or the like, or combinations thereof. In one embodiment, the alcohol is tert-butyl alcohol at about 97% w/w.

Adhesion layer 106, just below spread layer 108, can include an adhesion substance, a surfactant, and/or a solvent. The Fru-α-ValHis dipeptide created in the spread layer can pass readily through adhesion layer 106.

In some embodiments, the adhesion substance can serve to promote adhesion between spread layer 108 and masking layer 104. In one embodiment, the adhesion substance is poly-isopropylacrylamide (l100). In other embodiments, the adhesion substance can be polyvinylpyrrolidone (PVP). In some embodiments, the PVP can have a k90 chain length, a k30 chain length, a k15 chain length, or a combination thereof.

The adhesion layer surfactant can serve as a coating aid. In some embodiments, the adhesion layer surfactant is an octylphenol ethoxylate such as TRITON X-100. In some embodiments, the adhesion substance can be a combination of poly-isopropylacrylamide and octylphenol ethoxylate.

The solvent in adhesion layer 106 can be ethanol, isopropyl alcohol, methanol, t-butyl alcohol, acetone, or a combination thereof. In one embodiment, the adhesion layer solvent can be acetone. In some embodiments, when ethanol is used, no surfactant may be required.

Masking layer 104 can include a gel, at least one buffer, a pigment substance, a dispersing agent, a surfactant, a hardener, and/or a solvent/diluent. In some embodiments, the gel is a gelatin and/or a hardened gel.

The gel can promote layer cohesion, promote capillary force upon rewet, and/or provide a size exclusion mechanism. In some embodiments, the size exclusion mechanism can exclude high molecular weight interferents (upon crosslinking/hardening).

In some embodiments, the gel is a hardened gel. In one embodiment, the gel is Gel-RC Rousselot Dub Pig Dia Type 56 or 275 Bloom Type A NF Porcine Skin Gelatin.

In some embodiments, the pigment substance can create a reflective portion within the masking layer. The pigment substance can provide a white reflective surface and act to trap or mask interferents such as heme byproducts, catalase, and triglycerides. In some embodiments, the pigment substance can include a metal substance or metal. In some embodiments, the metal is titanium such as, but not limited to, titanium dioxide (TiO₂). The titanium dioxide can be an anatase titanium dioxide pigment with high whiteness and blue tone. In some embodiments, the pigment substance is Hombitan LC-S, Huntsman TiO₂, or Kemiera 300. In other embodiments, the titanium dioxide can be in other crystalline forms such as, but not limited to, rutile, brookite, akaogiite, and combinations thereof, or combinations with anatase.

In some embodiments, titanium dioxide and a hardened gel can act together to create a sieve where small molecular weight species (such as Fru-alpha-ValHis) can pass through the layer readily while larger molecular weight proteins (hemoglobin, catalase, protease) are excluded. In one embodiment, a sieve is created that allows Fru-alpha-ValHis to pass through.

In some embodiments, this exclusion of larger molecular weight proteins can be an essential feature of the masking layer as hemoglobin can create optical interference in the HbA1c measurement at 670 nm, catalase can consume peroxide necessary for dye oxidation, and/or protease can digest registration enzymes present in the gel layer (fructosyl peptide oxidase, horseradish peroxidase).

In some embodiments, the titanium dioxide can provide a uniform reflective surface which is used to reflect light from an analyzer light source to a detector for signal quantitation. The analyzer light source can be a light emitting diode (LED) or other light source that can provide light at the wavelengths described herein. After light contact or otherwise interacts with a sample, a sensor(s) can be used to read the amount of light a one or more wavelengths. The sensor can be a photo multiplier tube, a contact-image sensor, an image capturing sensor matrix, or a combination thereof.

Buffering the masking layer can serve to keep the masking layer at a desired pH. A desired pH can be about 6.0 to about 7.0, about 6.2 to about 7.2, about 6.5 to about 7.5, about 6.0 to about 8.0, about 7.0 to about 8.0, about 6.8 to about 7.2, about 7.4, about 7.2, about 7.0, or about 6.8.

In one embodiment, the at least one masking layer buffer can include a first buffer and a second buffer, each of which can be an acid salt or a base salt as required. In one embodiment, the first buffer is 3-(N-morpholino)propanesulfonic acid (MOPS). In one embodiment, the second buffer is beta,beta-dihydroxyl-1,4-piperazine dipropane sulfonic acid disodium salt (POPSO).

The dispersing agent can be a sodium polymethacrylate. The agent can be effective for rapid dispersion of pigments. In one embodiment, the dispersing agent is Daxad 30S.

The masking layer surfactant can act as a coating aid. The masking layer surfactant can be an anionic surfactant such as a polyether sulfonate. In one embodiment, the masking layer surfactant is TRITON X200E.

The hardener can serve to crosslink the gel in the gel layer and/or promote layer cohesion. In some embodiments, the hardener is bis(vinylsulfonylmethyl) (BVSM).

In some embodiments, the masking layer solvent/diluent is water.

Masking layer 104 can separate functional areas of the slide. For example, masking layer 104 can separate spread layer 108 from gel layer 102. This can separate the red blood cell lysis, denaturation/digestion of hemoglobin and liberation of Fru-α-ValHis dipeptide occurring in the spread layer from the Fru-α-ValHis dipeptide de-glycation and HRP/dye reaction to create colorimetric signal occurring in gel layer 102.

In some embodiments, a masking layer is not present. For example, when forming a slide where measurement of Hb at 540 nm is required, the titanium dioxide is not present because it would block the ability to read the hemoglobin.

However, in other embodiments, where measurement of Hb at 540 nm is required, masking layer 104 may still include titanium dioxide. In some embodiments, a reflected signal can be created in the gel layer for Fru-α-ValHis at 670 nm and a reflected signal can be created above masking layer for Hb at 540 nm that has not passed through masking layer. In such an embodiment, two separate reflected signals are measured, one from above to measure Hb at 540 nm and one from below to measure Fru-α-ValHis at 670 nm. These values can be used to determine a derived % HbA1c.

Gel layer 102 can include a gel, a buffer, at least one surfactant, a coupler solvent, a reductant, a detection agent, a cofactor, an amplification substance or catalyst, an oxidase, a hardener, and a solvent/diluent.

In some embodiments, the gel layer gel is a gel or gelatin. The gel can promote layer cohesion, promote capillary force upon rewet, and/or provide a size exclusion mechanism. In some embodiments, the size exclusion mechanism can exclude high molecular weight interferents (upon crosslinking/hardening). In some embodiments, the gel is a cross-linked gel. Crosslinking can improve layer integrity and decreased pore size to filter out additional interfering substances.

In one embodiment, the gel is Gel-32 TCGIII DI Gelatin. The gel can be porous.

In some embodiments, the gel can serve as a size exclusion mechanism.

The gel layer buffer can serve to keep the layer at a desired pH. A desired pH can be about 6.0 to about 7.0, about 6.2 to about 7.2, about 6.5 to about 7.5, about 6.0 to about 8.0, about 7.0 to about 8.0, about 6.8 to about 7.2, about 7.4, about 7.2, about 7.0, or about 6.8. The buffer can be an acid or a base as required. In one embodiment, the buffer is 3-(N-morpholino)propanesulfonic acid (MOPS).

Gel layer 102 can include one or more surfactants. The first surfactant can serve as a coating aid. In some embodiments, the gel layer's first surfactant is an octylphenol ethoxylate such as TRITON X-100.

The second surfactant can be used for dye dispersion. In one embodiment, the second surfactant is an alkylated sodium naphthalene sulfonate such as Alkanol XC.

The dye layer coupler solvent can be 2,4-di-n-pentyl phenol (KS-52) and/or 2,4-di-tert-pentyl phenol (KS-41).

A reductant can be added to prevent spurious dye oxidation. In one embodiment, the reductant can be 5,5-dimethyl-1,3-cyclohexanedione (Dimedone).

The detection agent can be a dye. The detection agent can be used for colorimetric signal generation. Virtually any dye can be used that provides a detectable signal. The dye can be N-carboxymethylaminocarbonyl)-4,4′-bis(dimethylamino)-diphenylamine sodium (DA-64), N,N,N′N′,N″,N″-hexa(3-sulfopropyl)-4,4′,4″-triamino-triphenylmethane hexasodium salt (TPM-PS), 10-(carboxymethylaminocarbonyl)-3,7-bis(dimethylamino)-phenothiazine sodium (DA-67), 2-(3,5-Dimethoxy-4-hydroxyphenol)-4,5-bis-(4-dimethylamino phenyl) imidazole, or a combination thereof. In one embodiment, the dye is 2-(3,5-dimethoxy-4-hydroxyphenol)-4,5-bis-(4-dimethylamino phenyl)imidazole.

The oxidase can be an oxidase that produces peroxide from Fru-alpha-ValHis. The oxidase can de-glycate Fru-alpha-ValHis to produce the peroxide. In one embodiment, the oxidase can be a fructosyl peptide oxidase.

In some embodiments, gel layer 102 can include an oxidase reaction cofactor. The cofactor can be a non-protein chemical compound that aids in the oxidase activity. In other embodiments, the cofactor can be flavin adenine dinucleotide (FAD), nicotinamide adenine dinucleotide (NAD), and/or coenzyme A (CoA). In one embodiment, the cofactor can be flavin adenine dinucleotide (FAD).

The amplification substance or catalyst can be any molecule that amplifies a colorimetric dye response. The peroxide produced from the Fru-alpha-ValHis oxidase reaction can interact with the dye whose signal is amplified by the amplification substance. In some embodiments, the amplification substance is a peroxidase such as, but not limited to, horseradish peroxidase (POD).

The hardener can serve to crosslink the gel in the gel layer and/or promote layer cohesion. In some embodiments, the hardener is bis(vinylsulfonylmethyl) (BVSM).

In some embodiments, the gel layer solvent/diluent is water.

In some embodiments, in gel layer 102, Fru-α-ValHis is de-glycated by the fructosyl peptide oxidase. The fructosyl oxidase is specific for both Fru-α-ValHis and Fru-α-Val. However, Fru-α-Val is not produced by the proteolysis of the hemoglobin beta chains. The specificity of the fructosyl oxidase can also prevent assay interference from other glycated proteins such as albumin.

The de-glycation reaction results in the production of peroxide by the cycling of the FAD cofactor. The horseradish peroxidase and peroxide oxidize the dye to a colored product which absorbs light at 670 nm.

In some embodiments, the hardened gel in gel layer 102 is an additional protective layer for exclusion of larger molecular weight proteins (hemoglobin, catalase, protease). Cross-linking the gel reduces the pore size of the layer and also helps to prevent dye particles from the gel layer from mixing with the masking layer (e.g., melt) components during coating.

In some embodiments, hardening of gel layer 102 can increase signal for the microslide. The hardening can increase signal by between about 5% and about 10%, between about 1% and about 10%, between about 1% and about 5%, between about 5% and about 20%, or between about 1% and about 20%.

In some embodiments, the interference prevention agent can be an ascorbic acid oxidase. This oxidase can react with ascorbic acid (vitamin C) in a sample to prevent the ascorbic acid from interfering with a measurement. In some embodiments, ascorbic acid in a sample may react with dyes in the gel layer to reduce them and diminish their color. This color reduction can result in a negative % A1c prediction bias, or simply a falsely low % A1c result. By including ascorbic acid oxidase, ascorbic acid in a sample can be eliminated thereby preventing negative prediction bias.

In some embodiments, samples can include mega doses of ascorbic acid from patients using large doses of the vitamin to alleviate or reduce symptoms of certain conditions. Without an interference prevention agent, such as ascorbic acid oxidase, results may be inaccurate. Thus, in some embodiments, slides as described herein include an interference prevention agent in the gel layer or any other appropriate layer of the slide.

In some embodiments, the microslide's dyes are analyzed using a detection paradigm. The detection paradigm can be by one or more reflective measurements. In one embodiment, reflective light measurement is used to detect absorbance by the dyes described herein.

In one embodiment, light at particular wavelengths are directed at the detection area 128 and the reflected light or reflective density is measured at a particular wavelength. Reflectance density (DR) is determined from reflectance. Reflectance density is equal to the Log of the inverse of reflectance.

In some embodiments, light is reflected off the titanium dioxide layers in the microslide. In some embodiments, the particular wavelengths of light can be 540 nm, 670 nm, or both. In some embodiments, wavelengths around these values can be used or ranges including these values can be used depending on signal strength and/or interferents that may absorb light in the same spectrum.

In some embodiments, wavelengths in the Soret band and Q band regions can be used. Wavelengths can include those at about 540 nm, such as, but not limited to about 535 nm, about 536 nm, about 537 nm, about 538 nm, about 539 nm, about 541 nm, about 542 nm, about 543 nm, about 544 nm, or about 545 nm can be used. In other embodiments, ranges of wavelengths can be used such as, but not limited to, a range of 530 nm to 540 nm, a range of 539 nm to 541 nm, a range of 538 nm to 542 nm, a range of 537 nm to 543 nm, a range of 536 nm to 544 nm, a range of 535 nm to 545 nm, a range of 540 nm to 545 nm, a range of 535 nm to 540 nm, a range of 535 nm to 575 nm, a range of 530 nm to 575 nm, a range of 540 nm to 575 nm, a range of 550 nm to 575 nm, or a range of 560 nm to 575 nm.

Likewise, in some embodiments, wavelengths at about 670 nm, such as, but not limited to about 665 nm, about 666 nm, about 667 nm, about 668 nm, about 669 nm, about 671 nm, about 672 nm, about 673 nm, about 674 nm, or about 675 nm can be used. In other embodiments, ranges of wavelengths can be used such as, but not limited to, a range of 660 nm to 680 nm, a range of 669 nm to 671 nm, a range of 668 nm to 672 nm, a range of 667 nm to 673 nm, a range of 666 nm to 674 nm, a range of 665 nm to 675 nm, a range of 670 nm to 675 nm, a range of 665 nm to 670 nm.

In one embodiment, reflective density is read by an automated analyzer such as, but not limited to, a VITROS analyzer. Endpoint reflective density or rates may be quantitated from the dye produced by this oxidation reaction.

Assay time can vary depending on analytical protocol or instrument being utilized. However, generally, the time from sample application through the enzymatic cascade to reflective density quantification is about 5 min to about 10 min, about 4 min to about 6 min, about 3 min to about 7 min, about 2 min to about 8 min, less than about 10 min, less than about 9 min, less than about 8 min, less than about 7 min, less than about 6 min, or less than about 5 min. In one embodiment, typical assay time is about 5 min at 37° C. on a VITROS analyzer.

Although the assay can be run at 37° C. on a VITROS analyzer, other temperatures can be used. For example, in some embodiments, the assay can be run at room temperature or at temperatures above or below 37° C.

In some embodiments, components of the enzymatic cascade such as protease, fructosyl oxidase, peroxidase, dye, and/or FAD may be removed from the gel layer as they are not needed for reading a hemoglobin signal at 540 nm.

In some embodiments, a single microslide can be used to measure only % A1c. In other embodiments, separate microslides can be used to individually measure Hb and HbA1c, respectively yielding a derived % A1c result. Also, all components necessary to determine the Hb concentration and HbA1c concentration may be incorporated in a single microslide to yield a derived % A1c result.

The devices, systems, and methods described herein can utilize whole blood patient samples without dilution or pretreatment. In some embodiments, the whole blood is unlysed. The use of whole blood saves time and resources when compared to testing systems that require processed blood.

In some embodiments, the devices, systems, and methods described herein can use small volumes of blood to measure HbA1c values. In clinical and diagnostic environments, blood sample volume can be critical especially when large panels of tests are being performed.

Small volumes of blood can be between about 1 μL and about 10 μL, between about 2 μL and about 8 μL, between about 4 μL and about 6 μL, between about 4 μL and about 5 μL, between about 4 μL and about 10 μL, between about 2 μL and about 5 μL, less than about 10 μL, less than about 8 μL, less than about 6 μL, or less than about 5 μL.

In some embodiments, the devices, systems, and methods described herein can measure HbA1c values in short amounts of time when compared to conventional methods. This can be referred to as rapid assay time. In clinical and diagnostic environments, measurement time can be critical when considering the cost of time and instrument throughput.

Rapid assay time can be about 1 min to about 10 min, about 2 min to about 8 min, about 3 min to about 7 min, about 4 min to about 7 min, about 4 min to about 8 min, about 5 min to about 7 min, about 6 min to about 8 min, about 5 min to about 8 min, less than about 10 min, less than about 8 min, less than about 7 min, or less than about 6 min.

With a rapid assay time, high throughput systems utilizing the herein described assays can run more assays per time period thereby generating more profit per time period than conventional assays. In some embodiments, a high throughput system can run between about 300 tests/hour and about 400 tests/hour, between about 350 tests/hour and about 400 tests/hour, between about 350 tests/hour and about 450 tests/hour, between about 300 tests/hour and about 500 tests/hour, between about 300 tests/hour and about 600 tests/hour, at least about 300 tests/hour, at least about 350 tests/hour, at least about 375 tests/hour, or at least about 400 tests/hour.

The devices, systems, and methods described herein can have assay specificity ensured by production of a substrate (Fru-alpha-ValHis) by proteolysis of the N-terminal beta chains of hemoglobin and de-glycation by a specific fructosyl peptide oxidase.

In some embodiments, the devices, systems, and methods do not suffer from hemoglobin structural variant (HbS, HbC) interference. Some commercially available assays suffer from HbS, HbC interference because they are antibody-based methods.

Methods of using the herein described microslides are also described.

In one embodiment, a single-slide method for detecting hemoglobin and glycated hemoglobin is described. This method can include contacting a spread layer of a microslide as described herein slide with a blood sample comprising red blood cells. The lysing agent releases glycated hemoglobin from the red blood cells, the denaturing agent contacts the glycated hemoglobin to denature the glycated hemoglobin, and the protease releases a fructosyl peptide from the denatured glycated hemoglobin. Then, the fructosyl peptide reaches said gel layer and contacts the fructosyl oxidase to release peroxide. The peroxidase and the peroxide contact the detection agent to release and/or generate a detectable signal.

In some embodiments, an interference prevention agent reacts with any ascorbic acid in the blood sample to prevent sample bias. In some embodiments, the interference prevention agent is ascorbic acid oxidase.

The amount of hemoglobin from blood sample is measured. The measuring comprises reading the reflectance density of the sample using a first wavelength of light. Also, the amount of glycated hemoglobin from the blood sample is measured. The glycated hemoglobin measurement comprises detecting the reflectance density of the detectable signal from the sample at a second wavelength of light. In some embodiments, the second wavelength of light is different from the first wavelength of light. In one embodiment, the first wavelength of light is 540 nm and the second wavelength of light is 670 nm.

In some embodiments, the first wavelength of light can be measured relatively early in the analysis and the second wavelength of light can be measured later in the analysis after a lag time. Lag time can be about 30 sec, about 40 sec, about 50 sec, about 60 sec, about 2 min, about 3 min, about 4 min, between about 30 sec and about 1 min, between about 40 sec and about 1 min, between about 30 sec and about 2 min, between about 30 sec and about 3 min, or between about 30 sec and about 4 min. This lag time allows sufficient time for the reaction cascade to occur.

In another embodiment, a single-slide method for direct detection of glycated hemoglobin is described. The method can comprise contacting the first film layer of a microslide as described herein with a blood sample comprising red blood cells. The lysing agent releases glycated hemoglobin from the red blood cells, the denaturing agent contacts the glycated hemoglobin to denature the glycated hemoglobin, and the protease releases a fructosyl peptide from the denatured glycated hemoglobin. Then, the fructosyl peptide traverses the second film layer, wherein the fructosyl peptide reaches the third film layer and contacts the fructosyl oxidase to release peroxide. The peroxidase and the peroxide react with the detection agent to produce a detectable signal.

In some embodiments, an interference prevention agent reacts with any ascorbic acid in the blood sample to prevent sample bias. In some embodiments, the interference prevention agent is ascorbic acid oxidase.

Then, the amount of glycated hemoglobin from said blood sample is measured. In such an embodiment, a measurement of non-glycated hemoglobin is not measured. The glycated hemoglobin measurement comprises detecting the reflectance density of the detectable signal in the sample. The glycated hemoglobin measurement comprises detecting the reflectance density of the detectable signal from the sample at a wavelength of light. In some embodiments, the wavelength of light is 670 nm.

In some embodiments, the herein described microslides can have a low unit manufacturing cost when compared to conventional HbA1c measurement assays. The microslides can reduce manufacturing cost by about 5% to about 10%, about 5% to about 20%, or about 10% to about 20%.

The herein described microslides can be produced by coating successive thin film layers over a transparent support. Thus, a microslide can be produced by applying a gel layer coating on the support (114), then a masking layer on the gel layer, an adhesion layer on the masking layer, and a spread layer on the adhesion layer.

In other embodiments, once a coating is formed by successive film layer deposition, the coating is slit to the appropriate width. Then, the slit coatings are assembled into finished microslides by chopping the slits into individual slide-sized chips which can be mounted along with a spacer web into an upper and lower slide mount. This process can be conducted on slide assembly machines (SAMs).

Individual slides can be packaged into carts for use on mainframe analyzers. Carts can include any number of slides appropriate for the analyzer. In some embodiments, the carts can have 50 microslides, 100 microslides, 200 microslides, at least 10 microslides, at least 15 microslides, at least 20 microslides, at least 50 microslides, or at least 100 microslides. In other embodiments, the carts can have 18 microslides, 50 microslides, or 60 microslides.

In some embodiments, spread layer components can be added using an inkjet deposition process. The spread layer components added by an inkjet deposition process can include the protease, the sodium nitrite, and/or the calcium chloride.

In some embodiments, a microslide can include a spacer web 130 between support layer 114 and lower slide mount 112. The spacer we can prevent microslide damage during assembly, particularly during welding of the upper and lower slide mounts.

In some embodiments, a microslide has a thickness. The thickness of the microslide including the spread layer, the adhesion layer, the masking layer, the gel layer, the optional spacer web and the support layer is about 100 μm, about 200 μm, about 300 μm, about 400 μm, about 500 μm, or about 600 μm.

### EXAMPLE 1

### Use of a TiO₂ Masking Layer to Reduce Hemoglobin Optical Interference

Six microslides are provided. Four of the microslides include TiO₂ in the masking layer and two do not. A sample of blood is dropped onto each slide on the spread layer.

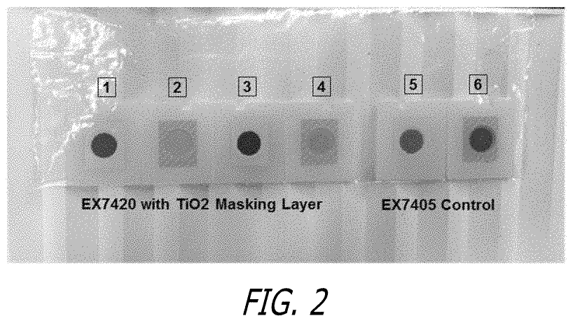

FIG. 2 illustrates the impact of the TiO₂ masking layer pictorially. Slide 1 and Slide 3 show the spot side of the TiO₂ masking layer slide. Hemoglobin is evident on the spot side of the slides. Slide 2 and Slide 4 show the read side of the TiO₂ masked slides. Hemoglobin is excluded from the gel layer (read side) of the slides.

In contrast, Slide 5 and Slide 6 respectively show the spot side (Slide 5) and read side (Slide 6) of a microslide not including TiO₂ in the masking layer. Hemoglobin is readily visible on Slide 6 (read side).

Thus, including TiO₂ in the masking layer prevents any substantial amount of hemoglobin from penetrating into the gel layer.

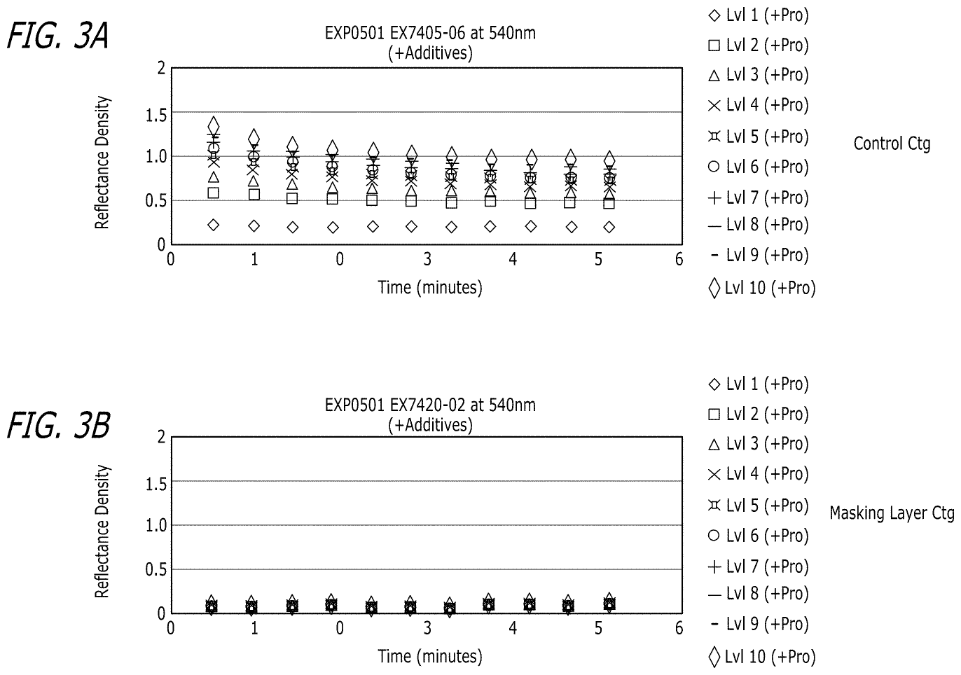

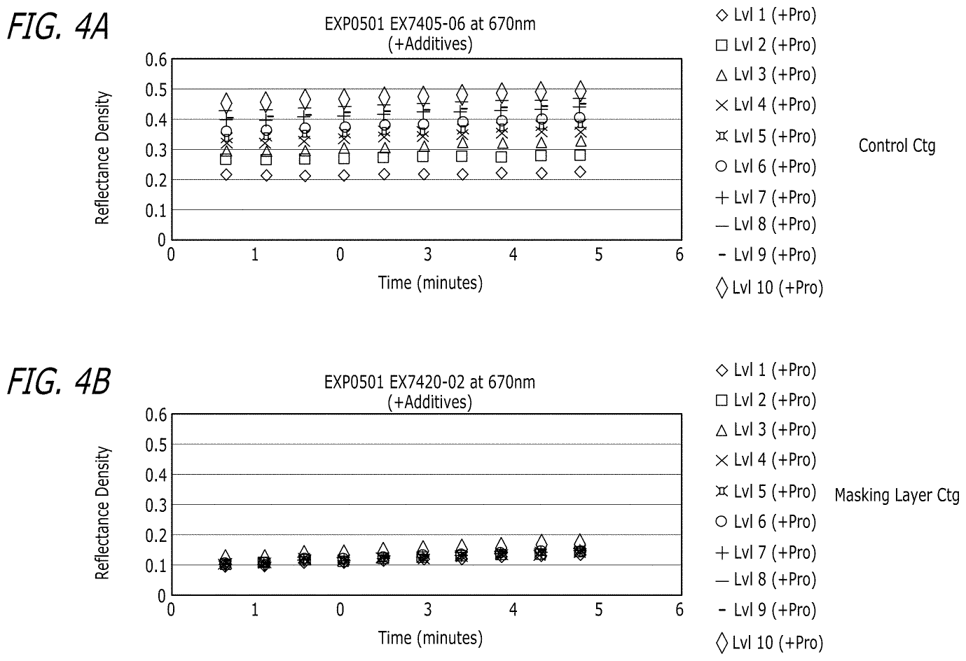

FIG. 3A and 3B and FIG. 4A and 4B show kinetic plots for a 10 level hemolysate series ranging in concentration from about 6 g/dL to about 20 g/dL hemoglobin for a “control coating” (non-TiO₂ masking layer) and a TiO₂ masking layer.

Kinetics are recorded over a 5 minute period at 540 nm and 670 nm. Note the differing scales on the plots. The hemoglobin signal at 540 nm for the control coating shows a typical kinetic profile. The 540 nm response for the TiO₂ masking layer slide illustrates that the 540 nm signal has been masked by exclusion of hemoglobin from the gel layer of the coating.

Similarly, the hemoglobin signal at 670 nm for the control coating shows a typical kinetic profile. This data shows that there is differential hemoglobin signal at 670 nm dependent on the concentration of hemoglobin being evaluated. This signal may lead to optical interference in the 670 nm dye read for the HbA1c assay requiring a correction algorithm. The TiO₂ in the masking layer greatly reduces the 670 nm hemoglobin signal, preventing optical interference in the HbA1c colorimetric assay (read at 670 nm).

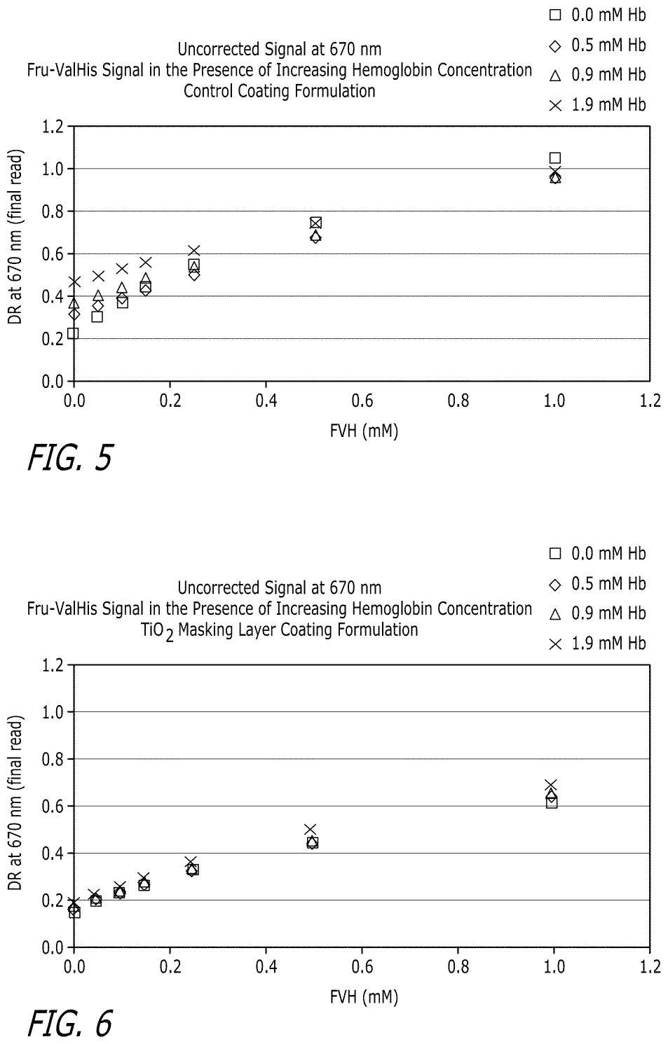

FIG. 5 and FIG. 6 illustrate dose response plots of pure Fru-α-ValHis dipeptide in fluids with increasing hemoglobin concentration comparing TiO₂ containing masking layer slides and masking layers not containing TiO₂. In FIG. 5 the fluids are run on slide not containing TiO₂. The data shows that as hemoglobin concentration increases, background signal at 670 nm (0.0 mM Fru-α-ValHis) increases resulting in loss of delta signal range across the Fru-α-ValHis levels tested. In FIG. 6 the fluids are run on TiO₂ containing masking layer slides. The data shows that as the hemoglobin concentration increases, background signal at 670 nm (0.0 mM Fru-α-ValHis) stays constant. Optical interference from hemoglobin at 670 nm has been reduced and/or eliminated.

### EXAMPLE 2

### HbA1c Microslide Test

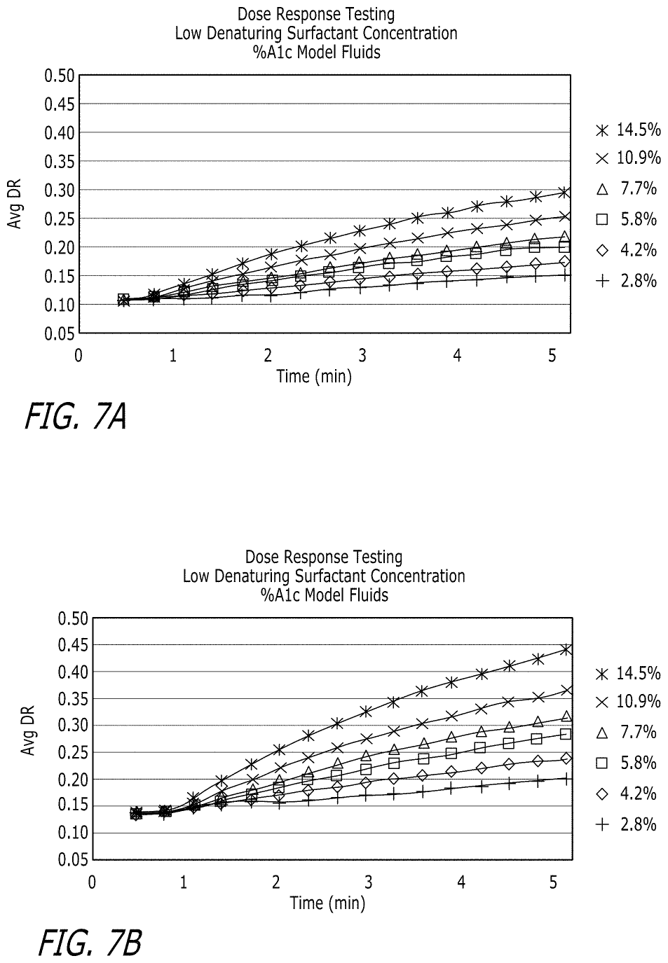

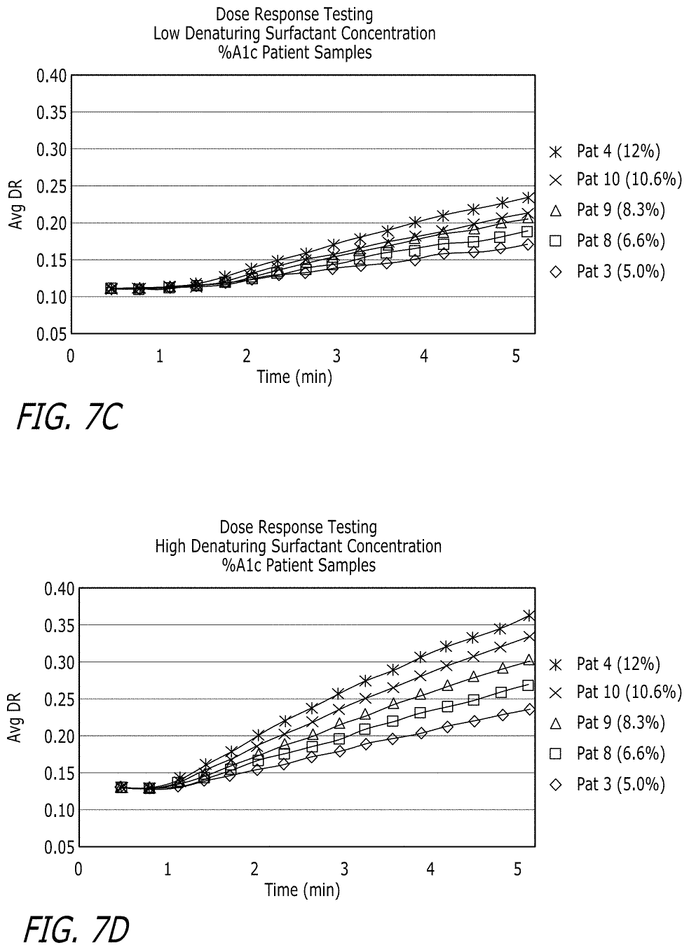

HbA1c microslide data for model % A1c fluids and whole blood patient samples are evaluated. FIGS. 7A-D illustrate reflectance density (DR) kinetic data at 670 nm for a comparison of model % A1c fluid signal generation and % A1c patient sample signal generation.

The % A1c model fluids and % A1c patient samples are of comparable % A1c. All HbA1c microslide components are x-hopper coated with the exception of sodium nitrite and protease which are applied by an inkjet deposition process.

Also, % A1c model fluid and % A1c patient sample kinetic responses are increased by coating a higher concentration (coverage) of the denaturing surfactant (N-lauroylsarcosine, NLS). As can be seen in FIGS. 7A-D, % A1c patient samples have a similar kinetic profile to the % A1c model fluids. Also, FIGS. 7A-D show that there is good discrimination between the % A1c levels evaluated.

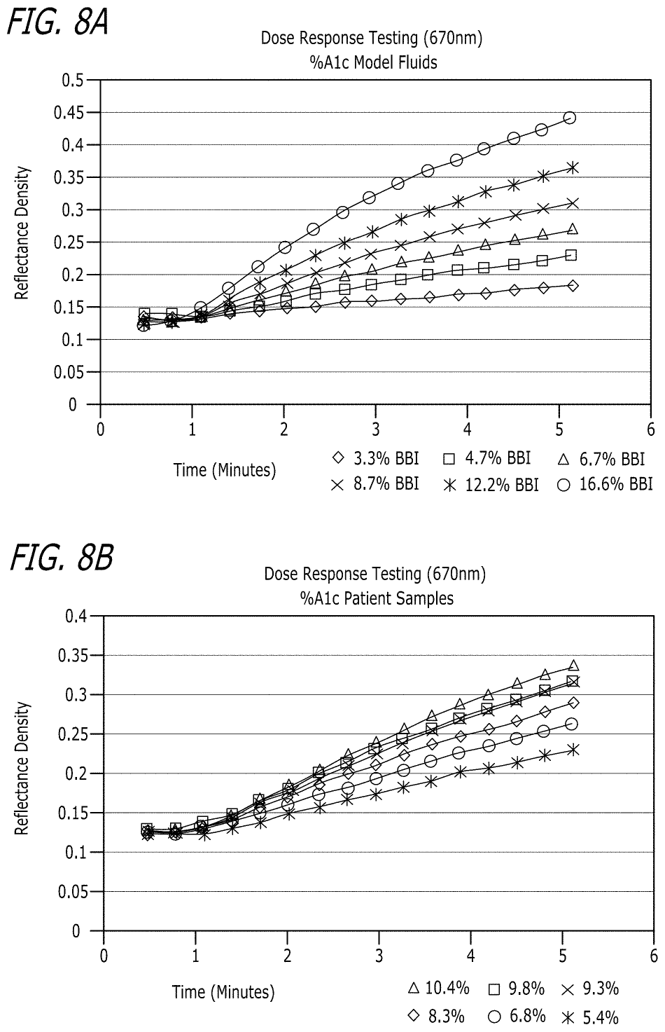

FIGS. 8A and 8B illustrate DR kinetic data at 670 nm for a comparison of model % A1c fluid signal generation and % A1c patient sample signal generation. This data is generated with an x-hopper coating which incorporated all the components to conduct the herein described enzymatic cascade.

The % A1c model fluids and % A1c patient samples are of comparable % A1c. As can be seen in FIGS. 8A and 8B, % A1c patient samples have a similar kinetic profile to the % A1c model fluids. Also, FIGS. 8A and 8B show that there is good discrimination between the % A1c levels evaluated.

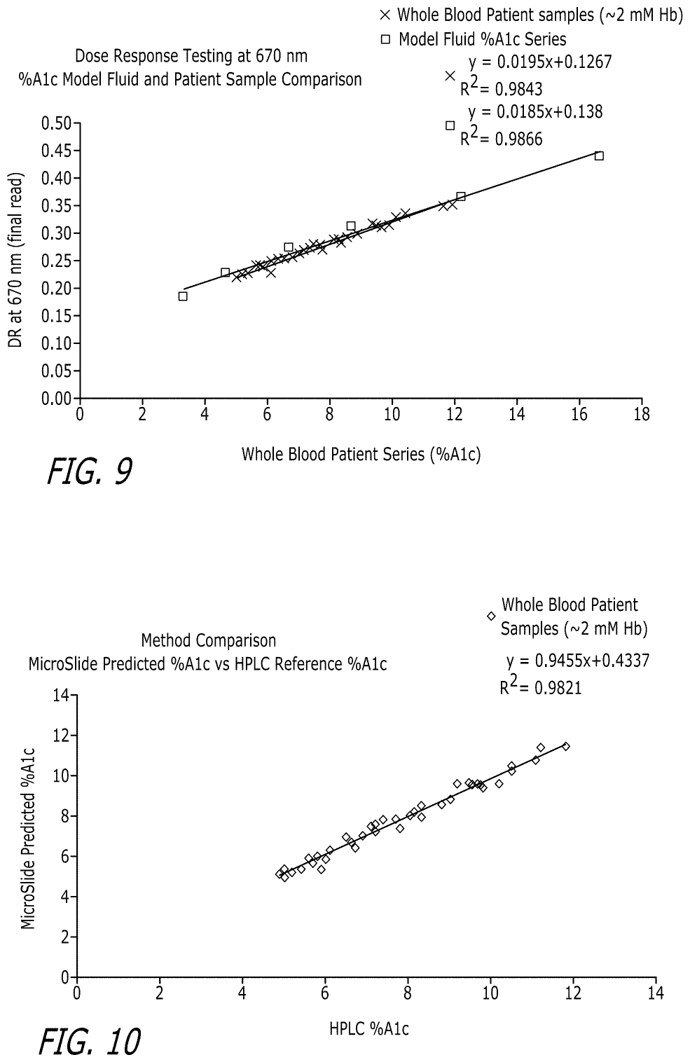

FIG. 9 illustrates DR dose response data at 670 nm for % Mc model fluids and whole blood % A1c patient samples. This data is generated using the same coating for the data shown in FIGS. 8A and 8B. This data clearly shows that whole blood patient samples give the same response curve as % A1c model fluids made from purified glycated hemoglobin. This indicates that the herein described microslide is able to lyse the red blood cells, denature and digest the glycated hemoglobin producing the substrate which results in a colorimetric signal.

FIG. 10 illustrates a correlation plot for patient sample predicted % A1c results for the HbA1c MicroSlide versus the assigned HPLC % A1c values. A linear calibration model based on MicroTip % A1c reference and HbA1c microslide reflectance density at 670 nm is used to predict each of the 6 microslide replicates for each patient sample. The mean of the microslide % A1c predictions is compared to the BioRad Variant High Performance Liquid Chromatography (HPLC) % A1c results. The microslide % A1c assay has a very strong correlation to the HPLC % A1c assay as is evidenced in FIG. 10.

Based on the data acquired and studied in this Example, a direct % A1c measurement is feasible with the herein described microslides.

### EXAMPLE 3

### Derived % A1c using HbA1c Component and Hemoglobin Component Determinations

Here HbA1c microslides as described herein are used in combination with Hemoglobin microslides to generate derived % A1c results for patient samples.

Here, the HbA1c microslide measures the component glycated hemoglobin while a separate Hemoglobin microslide measures the total hemoglobin component. The two results are used to calculate a derived % A1c test result.

FIG. 11 illustrates a Hemoglobin component dose response plot for the % Mc patient samples used in the previous section. The reflectance density at 540 nm is plotted versus the Vitros MicroTip Hemoglobin concentration result (in g/dL).

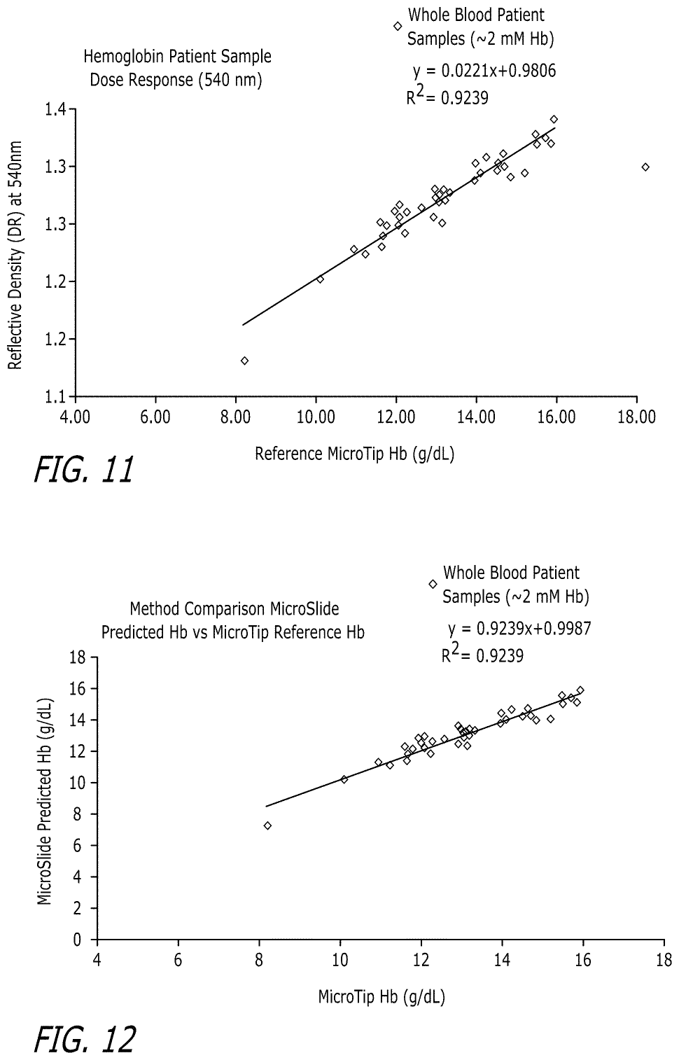

FIG. 12 illustrates a correlation plot for patient sample predicted microslide hemoglobin component concentration versus the assigned MicroTip reference hemoglobin values. A linear calibration model based on the MicroTip hemoglobin reference and hemoglobin microslide reflectance at 540 nm is used to predict each of the 6 Hemoglobin microslide replicates for each patient sample (FIG. 11). The mean of the microslide hemoglobin component predictions is compared to the MicroTip hemoglobin reference value. The microslide hemoglobin component assay has a good correlation to the MicroTip hemoglobin assay.

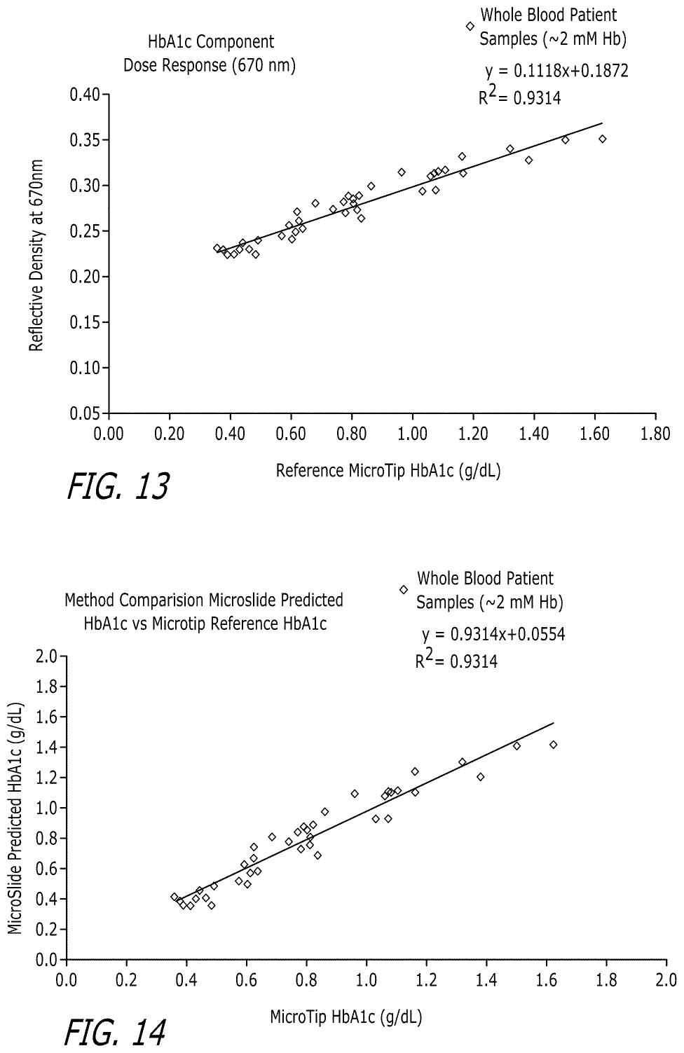

FIG. 13 illustrates a HbA1c component dose response plot for the % A1c patient samples used in Example 2. The reflectance density at 670 nm is plotted versus the MicroTip HbA1c component concentration result (in g/dL).

FIG. 14 illustrates a correlation plot for patient sample predicted microslide HbA1c component concentration versus the assigned MicroTip reference HbA1c component values. A linear calibration model based on the MicroTip HbA1c component reference value and HbA1c microslide reflectance at 670 nm is used to predict each of the 6 HbA1c component microslide replicates for each patient sample (FIG. 13). The mean of the microslide HbA1c component predictions is compared to the MicroTip HbA1c component reference value. The microslide HbA1c component assay has a good correlation to the MicroTip HbA1c component assay.

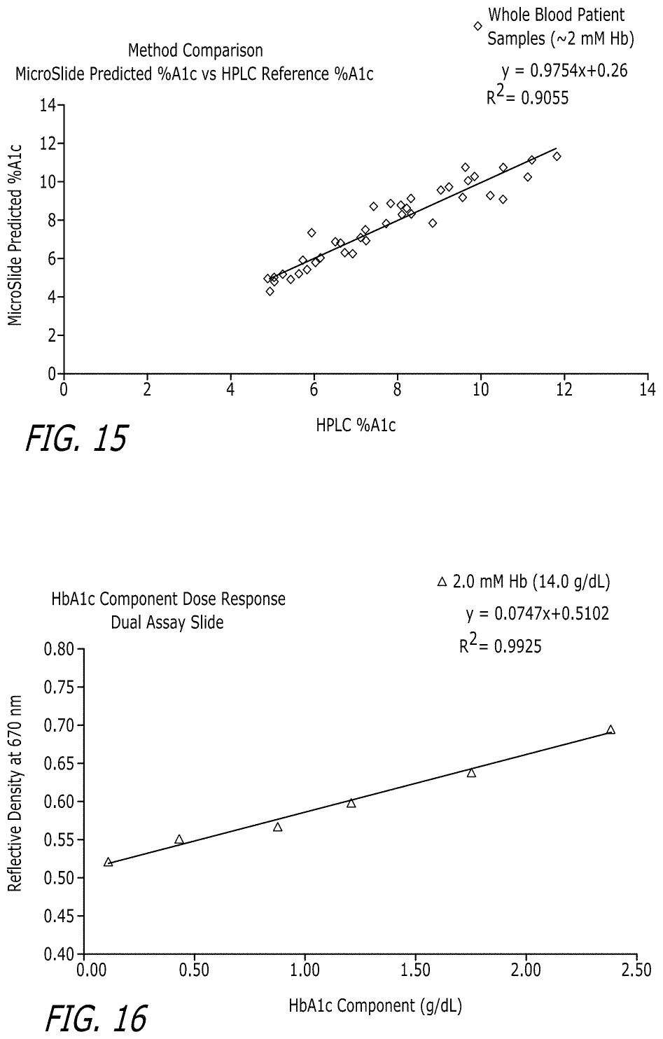

FIG. 15 illustrates a correlation plot for patient sample predicted MicroSlide derived % A1c versus the BioRad Variant HPLC reference % A1c values. The microslide % Mc values are obtained by using the hemoglobin component result (g/dL) and HbA1c component result (g/dL) to generate a derived % Mc value using the National Glycohemoglobin Standardization Program “master equation” (see equations below taken from the VITROS HbA1c MicroTip Assay Instructions for Use, Pub. No. J55871_EN, Version 2.0).

$$\frac{HbA 1 c}{SI Units}$$

$$HbA 1 c ( \frac{mmol}{mol} ) = \frac{HbA 1 c [ \frac{g}{dL} ]}{Hb [ \frac{g}{dL} ]} \times 1000$$

$$\frac{\% A 1 c}{NGSP Units}$$

$$\% A 1 c = ( {IFCC}^{*} \times 0.09148 ) + 2.152$$

$${}^{*} IFCC = HbA 1 c ( mmol / mol ) SI Units$$

The mean of the microslide % A1c values is compared to the HPLC % A1c reference value. The microslide derived % Mc assay has a good correlation to the HPLC % A1c assay.

The Example 3 data demonstrates that a derived % A1c measurement is feasible using a Hemoglobin microslide assay as described herein in combination with a HbA1c microslide assay to generate a derived % A1c result.

### EXAMPLE 4

### Derived % A1c using HbA1c Component and Hemoglobin Component Determinations

Here a single microslide test element is used to generate hemoglobin spectra results and HbA1c enzymatic results to generate derived % A1c results for patient samples. This is termed a “dual assay” microslide test element. The microslide used does not include titanium dioxide in the masking layer, because it would block the ability to read the hemoglobin signal at 540 nm. The protease and sodium nitrite may be incorporated into the microslide by inkjet deposition or the x-hopper coating process.

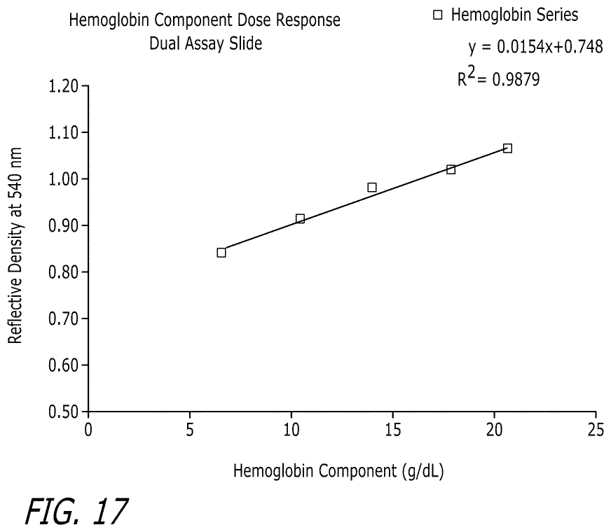

FIG. 16 and FIG. 17 illustrate HbA1c component dose response data (670 nm) and hemoglobin component dose response (540 nm) for a dual assay microslide test element. The reflective density (DR) dose response data shows that the dual assay microslide test element is able to register the BBI HbA1c fluid series in a dose dependent manner at 670 nm indicating that the enzymatic cascade is functional. The microslide test element also measures the hemoglobin spectra read at 540 nm in a dose dependent manner. As described in Example 3, a derived % Mc measurement is feasible using the “dual assay” microslide test element.

### EXAMPLE 5

### Direct % A1c Measurement

A sample of whole blood is applied to a microslide as described herein. The herein described enzymatic cascade produces an oxidized dye that absorbs light at 670 nm while the microslide filters out non-glycated hemoglobin using a masking layer. Light is reflected on the titanium dioxide in the masking layer and reflective density is read at 670 nm. Using a % Mc calibration curve, the reflective density is directly calculated as % A1c.

### EXAMPLE 6

### Derived % A1c using a Single Sample

A sample of whole blood is applied to a microslide as described herein that does not include titanium dioxide in the masking layer. The herein described enzymatic cascade produces an oxidized dye that absorbs light at 670 nm as well as allowing measurement of non-glycated hemoglobin at 540 nm. The values attained from measurements at 540 nm and 670 nm allow calculation of a derived % A1c.

### EXAMPLE 7

### Derived % A1c using Two Samples on a Single Microslide

Two samples of whole blood are applied to two separate areas of a microslide. One area includes titanium dioxide in its masking layer and the herein described enzymatic cascade produces an oxidized dye that absorbs light at 670 nm. The other sample area does not include titanium dioxide allowing direct measurement of non-glycated hemoglobin at 540 nm. The values attained from measurements at 540 nm and 670 nm allow calculation of a derived % A1c.

### EXAMPLE 8

### Derived % A1c using Two Measurements on a Single Slide

A sample of whole blood is applied to a microslide as described herein. The herein described enzymatic cascade produces an oxidized dye that absorbs light at 670 nm while the microslide negates non-glycated hemoglobin signal using its masking layer. Light is reflected from below the masking layer using the titanium dioxide and reflective density is read at 670 nm. Also, light is reflected from above the masking layer on the titanium dioxide and VtE beads in the spread layer and reflective density of the hemoglobin is read at 540 nm. The values attained from measurements at 540 nm and 670 nm allow calculation of a derived % A1c.

### EXAMPLE 9

### Reduction in Ascorbic Acid Interference

Two samples are run from a patent. This patient has mega dosed on ascorbic acid in order to achieve an anti-cancer affect. The first sample is run on a slide including ascorbic acid oxidase in its gel layer and a second sample is run on a slide without ascorbic acid oxidase.

The result from the first sample shows a higher % A1c value than the second sample.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A slide comprising:
a stack of film layers comprising, from bottom to top,
a first film layer comprising a cross-linked gel, wherein said cross-linked gel comprises a detection agent, a fructosyl oxidase, an interference prevention agent, and a peroxidase;
a second film layer comprising a first gel; and
a third film layer comprising a lysing agent, a denaturing agent and a protease.

2. The slide of claim 1, wherein said lysing agent is selected from the groups consisting of octylphenol ethoxylate (TRITON X-100), TWEEN (TWEEN 20), sodium dodecyl sulfate (SDS), cetyltrimethylammonium bromide (CTAB), tetradecyltrimethylammonium bromide (TTAB), polyoxyethylene lauryl ethers (POEs) and NONIDET P-40 (NP-40).

3. The slide of claim 1, wherein said denaturing agent is one or more of sodium nitrite or N-lauroylsarcosine (NLS).

4. The slide of claim 1, wherein said protease is a metalloproteinase, an endoprotease or an exoprotease.

5. The slide of claim 1, wherein said detection agent is selected from the group consisting of N-carboxymethylaminocarbonyl)-4,4′-bis(dimethylamino)-diphenylamine sodium (DA-64), N,N,N′N′,N″,N″-hexa(3-sulfopropyl)-4,4′,4″-triamino-triphenylmethane hexasodium salt (TPM-PS), 10-(carboxymethylaminocarbonyl)-3,7-bis(dimethylamino)-phenothiazine sodium (DA-67), and 2-(3,5-dimethoxy-4-hydroxyphenol)-4,5-bis-(4-dimethylamino phenyl) imidazole

6. The slide of claim 1, wherein said second film layer further comprises a reflective material portion.

7. The slide of claim 6, wherein said reflective material portion comprises titanium.

8. The slide of claim 1, wherein said third film layer further comprises a layer with particles having a diameter of about 25 μm.

9. The slide of claim 1, wherein said third film layer further comprises an a fructosyl oxidase specific for a Fru-α-ValHis peptide or a Fru-α-Val amino acid.

10. A single-slide method for detecting hemoglobin and glycated hemoglobin comprising:
a) providing a slide including
a stack of film layers comprising, from bottom to top,
a first film layer comprising a cross-linked gel, wherein said cross-linked gel comprises a detection agent, a fructosyl oxidase, an interference prevention agent, and a peroxidase;
a second film layer comprising a first gel; and
a third film layer comprising a lysing agent, a denaturing agent and a protease.
b) contacting said third film layer of said slide with an unlysed blood sample comprising red blood cells, wherein said lysing agent releases glycated hemoglobin from said red blood cells, wherein said denaturing agent contacts said glycated hemoglobin to denature said glycated hemoglobin, and wherein said protease releases a fructosyl peptide from the denatured glycated hemoglobin, wherein said fructosyl peptide reaches said first film layer and contacts said fructosyl oxidase to release peroxide, and wherein said peroxidase and said peroxide contact said detection agent to release a detectable signal
c) measuring the amount of hemoglobin from said blood sample, wherein said measuring the amount of hemoglobin comprises reading the reflectance density of the sample from said slide at a first wavelength of light; and
d) measuring the amount of glycated hemoglobin from said blood sample, wherein said measuring the amount of glycated hemoglobin comprises detecting the reflectance density of the detectable signal from said sample at a second wavelength of light, wherein said second wavelength of light is different from said first wavelength of light.

11. The method of claim 10, wherein said first wavelength of light is 540 nm and said second wavelength of light is 670 nm.

12. The method of claim 10, wherein said lysing agent is a detergent is selected from the group consisting of octylphenol ethoxylate (TRITON X-100), TWEEN (TWEEN 20), sodium dodecyl sulfate (SDS), cetyltrimethylammonium bromide (CTAB), tetradecyltrimethylammonium bromide (TTAB), polyoxyethylene lauryl ethers (POEs) and NONIDET P-40 (NP-40).

13. The method of claim 10, wherein said protease is a metalloproteinase, an endoprotease or an exoprotease.

14. The method of claim 10, further comprising contacting said fructosyl peptide with an oxidase cofactor is flavin adenine dinucleotide (FAD).

15. The method of claim 10, wherein said detection agent is a leuco-dye.

16. A single-slide method for direct detection of glycated hemoglobin comprising:
a) providing a slide including
a stack of film layers comprising, from bottom to top,
a first film layer comprising a cross-linked gel, wherein said cross-linked gel comprises a detection agent, a fructosyl oxidase, an interference prevention agent, and a peroxidase;
a second film layer comprising a first gel and a reflective material portion; and
a third film layer comprising a lysing agent, a denaturing agent and a protease.
b) contacting said third film layer of said slide with a blood sample comprising red blood cells, wherein said lysing agent releases glycated hemoglobin from said red blood cells, wherein said denaturing agent contacts said glycated hemoglobin to denature said glycated hemoglobin, and wherein said protease releases a fructosyl peptide from the denatured glycated hemoglobin, wherein said fructosyl peptide traverses said second film layer, wherein said fructosyl peptide reaches said first film layer and contacts said fructosyl oxidase to release peroxide, and wherein said peroxidase and said peroxide contact said detection agent to release a detectable signal; and
c) measuring the amount of glycated hemoglobin from said blood sample, wherein said measuring the amount of glycated hemoglobin comprises detecting the reflectance density of the detectable signal in said sample.

17. The method of claim 16, wherein said reflective material portion comprises a metal.

18. The method of claim 16, wherein said fructosyl peptide traverses said cross-linked gel.

19. The method of claim 16, wherein said protease is a metalloproteinase, an endoprotease or an exoprotease.

20. The method of claim 16, further comprising contacting said fructosyl peptide with an oxidase cofactor is flavin adenine dinucleotide (FAD).
